(19) United States
(12) Patent Application Publication
(10) Pub. No.: US 2009/0269421 A1
(43) Pub. Date: Oct. 29, 2009

(54) COATING MATERIAL

(75) Inventor: Shouji Ooya, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/147,879

(22) Filed: Jun. 27, 2008

(30) Foreign Application Priority Data
Jun. 29, 2007 (JP) 2007-171365

(51) Int. Cl. A61K 31/337; C09H 3/00; A61K 33/24; A61K 31/436; A61P 35/00; A61P 31/00; A61P 37/06; A61P 29/00

(52) U.S. Cl. 424/649; 530/354; 514/449; 514/291

(57) ABSTRACT

It is an object of the present invention to provide a coating material for a biomedical material surface which can control cell adhesiveness/non-cell adhesiveness and improve the biocompatibility of a material surface. The present invention provides a coating material for a surface of a biomedical material, which comprises a recombinant gelatin.

### FIELD OF THE INVENTION

The present invention relates to a coating material for a biomedical material surface, which comprises a recombinant gelatin.

### BACKGROUND ART

A variety of biomedical materials have been examined in the field of high-technology medical care represented by regenerative medicine. In particular, synthetic polymers and metal compounds are often used as materials for artificial blood vessels, artificial tissues, and artificial organs. However, in general, such materials are not designed for specific applications. In practice, general-purpose synthetic polymers that meet medical grade requirements are used.

In general, synthetic polymers have poor cell adhesiveness. Thus, in order to cover a material surface with vascular endothelial cells, a material surface is coated with a protein having high cell adhesiveness, or else such protein is fixed on a material surface. Collagens or gelatins that are denatured proteins obtained from collagens are generally used. However, even with the use of such materials, sufficient adhesion of vascular endothelial cells cannot be achieved. Thus, a material having improved cell adhesiveness has been required.

In addition, actions of a material on a living body are exhibited at the interface between such material and the living body. Therefore, it is very important in terms of material design to impart biocompatibility to a material surface.

In order to impart biocompatibility to a surface that comes into contact with blood, hydrophilization of the surface, fixation of antithrombogenic albumin or heparin to the surface, fixation of urokinase, which is a fibrinolytic protein, and coating with vascular endothelial cells are carried out.

Meanwhile, stents are used to prevent restenosis of blood vessels subjected to treatment of vascular diseases such as myocardial infarction. Nevertheless, restenosis often occurs and thus additional treatment is necessary. In recent years, drug-releasing stents have been gaining attention. Specifically, drug-releasing stents obtained by coating the outer surfaces thereof with materials comprising anticancer agents, immunosuppressive agents, or the like have been developed. With the use of such a stent, a blood vessel is not excessively restored, and thus blood vessel restenosis is prevented. It is necessary for a coating material for such stent to retain a drug and release the drug over a certain period of time, and further, to prevent cells from adhering thereto and growing thereon. However, no materials having high biocompatibility and such properties have yet been available in practice.

Further, since biopolymers extracted from living tissue are generally used, the following problems exist: (1): a living-body-derived infectious disease or inflammation caused by impurities might occur; (2): it is difficult to maintain stable material properties such as its molecular weight and its structure; and (3): it is difficult to accurately design a biopolymer by, for example, appropriately modifying a biopolymer in accordance with the application.

In recent years, along with the significant development of genetic engineering techniques, protein synthesis has been carried out by introducing genes into Escherichia coli or yeast. A variety of recombinant collagen-like proteins have been synthesized by such techniques (e.g., EP0926543B, WO02/052342, EP1063565B, WO2004/085473, EP1014176A, and U.S. Pat. No. 6,992,172). However, the application of such proteins in order to impart biocompatibility to biomedical material surfaces has not been examined.

### SUMMARY OF THE INVENTION

It is an object of the present invention to provide a coating material for a biomedical material surface which can control cell adhesiveness/non-cell adhesiveness and improve the biocompatibility of a material surface.

As a result of intensive studies in order to achieve the above object, the present inventors have found that cell adhesiveness can be effectively improved with the use of a recombinant gelatin as a coating material for a biomedical material surface. This has led to the completion of the present invention.

Thus, the present invention provides a coating material for a surface of a biomedical material, which comprises a recombinant gelatin.

Preferably, the recombinant gelatin has a homology of 80% or more with the amino acid sequence of a natural collagen.

Preferably, the recombinant gelatin comprises GXY triplets that are characteristic of collagen and has a molecular weight of 2 KDa to 100 KDa.

Preferably, the recombinant gelatin comprises GXY triplets that are characteristic of collagen and has a molecular weight of 10 KDa to 90 KDa.

Preferably, the recombinant gelatin comprises repeats of a partial sequence of a natural collagen.

Preferably, the biomedical material is a material used in vivo.

Preferably, the biomedical material is a material used for intravascular treatment.

Preferably, the biomedical material is a stent or an artificial blood vessel.

Preferably, the surface of a biomedical material is formed with segmented polyurethane, polyethyleneterephthalate, polylactic acid, polyglycolic acid, a lactic acid-glycol acid copolymer, stainless steel, or nickel.

Preferably, a drug is contained in the coating material of the present invention.

Preferably, the drug is an anti-inflammatory agent, an antibacterial agent, an antibiotic, an anticancer agent, or an immunosuppressive agent.

Preferably, the drug is an anticancer agent or an immunosuppressive agent.

Preferably, the drug is paclitaxel, docetaxel, cisplatin, rapamycin, or tacrolimus.

Preferably, the drug is a cytokine, a hormone, a polypeptide, or a nucleic acid.

Preferably, the recombinant gelatin is crosslinked.

Preferably, the crosslinking is carried out with the use of an aldehyde, a condensing agent, or an enzyme.

Preferably, an organic fluorine compound is used for inclusion of the drug or coating of the surface of a biomedical material.

The practice of the present invention enables: (1): control of cell adhesiveness; (2): design of biodegradability; (3): control of strength; and (4) design of tissue adhesiveness.

### BEST MODE FOR CARRYING OUT THE INVENTION

Hereinafter, embodiments of the present invention will be described in detail.

Examples of the recombinant gelatin used in the present invention include, but are not limited to, those described in EP1014176 A2, U.S. Pat. No. 6,992,172, WO2004-85473, and the like. In addition, biopolymers to be used may be partially hydrolyzed. The above gelatin may have an amino acid identity (with respect to a living-body-derived collagen sequence) of 40%, preferably 50% or more, more preferably 80% or more, and most preferably 90% or more. The term “collagen(s)” herein used refers to any naturally occurring collagen. However, such collagens are preferably type-I, II, III, IV, and V collagens and more preferably type-I, II, and III collagens. In another embodiment, such collagens are derived preferably from humans, bovines, pigs, mice, and rats, and more preferably from humans. The isoelectric point of the recombinant gelatin used in the present invention is preferably 5 to 10, more preferably 6 to 10, and further preferably 7 to 9.

Preferably, the recombinant gelatin has GXY triplets that are characteristic of collagen. The molecular weight thereof is 2 KDa to 100 KDa, preferably 2.5 KDa to 95 KDa, more preferably 5 KDa to 90 KDa, and most preferably 10 KDa to 90 KDa. Compared with other proteins, a GXY triplet that is characteristic of collagen is a highly specific partial structure in terms of amino acid composition and amino acid sequence of gelatin/collagen. Glycine corresponds to one-third of such partial structure. In the amino acid sequence, glycine is repeatedly found as every third amino acid. Glycine is the simplest amino acid, and thus the position thereof is not restricted in a molecular chain. Therefore, glycine significantly contributes to reproduction of a helix structure upon gel formation. Amino acids denoted by X and Y mainly contain imino acids (e.g., proline and oxyproline). Imino acids correspond to 10% to 45% of the total amino acids.

Preferably, the recombinant gelatin is not deaminated.

Preferably, the recombinant gelatin may or may not have procollagen.

Preferably, the recombinant gelatin is a substantially pure collagen material which is prepared with a nucleic acid encoding a natural collagen.

The recombinant gelatin used in the present invention is excellent in terms of biocompatibility and noninfectivity. In addition, the recombinant gelatin used in the present invention has a uniform structure compared with natural gelatins. Since the sequence of the recombinant gelatin is determined, it is possible to accurately design a recombinant gelatin with few variations in terms of strength and degradability via crosslinking or the like as described below.

In general, minimal amino acid sequences that function as cell adhesion signals in polypeptides have been known (e.g., “Pathophysiology” (Byotai Seiri), Vol. 9, No. 7 (1990), p. 527, published by Nagai Shuppan). Among them, the following sequences are preferable as sequences to which many types of cells can adhere, such sequences being each represented by a combination of amino acid alphabets: the RGD sequence, the LDV sequence, the REDV sequence (SEQ ID NO:6), the YIGSR sequence (SEQ ID NO:7), the PDSGR sequence (SEQ ID NO:8, the RYVVLPR sequence (SEQ ID NO:9), the LGTIPG sequence (SEQ ID NO:10), the RNIAEIIKDI sequence (SEQ ID NO:11), the IKVAV sequence (SEQ ID NO:12), the LRE sequence, the DGEA sequence (SEQ ID NO:13), and the HAV sequence. The RGD sequence, the YIGSR sequence (SEQ ID NO:7), the PDSGR sequence (SEQ ID NO:8), the LGTIPG sequence (SEQ ID NO:10), the IKVAV sequence (SEQ ID NO:12), and the HAV sequence are more preferable. The RGD sequence is particularly preferable. In view of cell adhesion/growth, the content of such minimal amino acid sequences in a single molecule is preferably 3 to 50 amino acid sequences, more preferably 4 to 30 amino acid sequences, and particularly preferably 5 to 20 amino acid sequences. In the case of the recombinant gelatin of the present invention, expression of such sequences is controlled such that desired cell adhesiveness can be achieved.

The type of natural collagen with which the recombinant gelatin has a high sequence homology is not particularly limited as long as the present invention can be carried out. In general, the type of a necessary sequence significantly differs depending on treatment application. That is, a sequence that is similar to the sequence of a collagen necessary for the corresponding tissue is desired. For instance, in a case of the surface of a material used for cartilage treatment, the sequence of a type-II collagen is desired. In a case of a blood vessel, it is desired that a type-I collagen be used for the outer membrane and a type-IV collagen be used for the inner membrane.

In a case in which the recombinant gelatin alone is not sufficient in terms of performance, the recombinant gelatin may be mixed with another material or may be formed into a complex with another material. For instance, the recombinant gelatins may be mixed with a different recombinant gelatin, a different biopolymer, or a different synthetic polymer. Examples of such biopolymer include polysaccharides, polypeptides, proteins, nucleic acids, and antibodies. Preferably, polysaccharides, polypeptides, and proteins are used. Examples of polysaccharides, polypeptides, and proteins include collagens, gelatins, albumin, fibroin, and casein. Such examples may be partially chemically modified according to need. For instance, hyaluronic acid ethyl ester may be used. Examples of polysaccharides include glycosaminoglycan represented by hyaluronic acid or heparin, chitin, and chitosan. Further, examples of polyamino acids include poly-γ-glutamic acid.

The recombinant gelatin used in the present invention can be chemically modified in accordance with applications. Chemical modification may include introduction of a low molecular compound or a different polymer (such as a biopolymer (e.g., sugar or protein), a synthetic polymer, or a polyamide) into a carboxyl group or an amino group in a side chain of a recombinant gelatin, crosslinking between recombinant gelatin chains, and the like. In the case of introduction of a low molecular compound into the recombinant gelatin, a carbodiimide-based condensing agent or the like may be used.

A crosslinking agent used in the present invention is not particularly limited as long as the present invention can be carried out. Such a crosslinking agent may be a chemical crosslinking agent or an enzyme. Examples of a chemical crosslinking agent include formaldehyde, glutaraldehyde, carbodiimide, and cyanamide. Preferred examples are formaldehyde and glutaraldehyde.

Further, crosslinking of a recombinant gelatin includes light irradiation of a gelatin into which a photoreactive group has been introduced and light irradiation in the presence of a photosensitizer. Examples of a photoreactive group include a cinnamyl group, a coumarin group, a dithiocarbamyl group, a xanthene dye, and camphorquinone.

When enzymatic crosslinking is carried out, an enzyme to be used is not particularly limited as long as it has an effect of crosslinking between recombinant gelatin chains. Transglutaminase and laccase are preferably used to carry out crosslinking, and transglutaminase is most preferably used. Specific examples of a protein that is subjected to enzymatic crosslinking with transglutaminase are not particularly limited, as long as such proteins each have a lysine residue and a glutamine residue. Transglutaminase may be derived from a mammal or a microorganism. Specific examples thereof include the Activa series by Ajinomoto Co., Inc.; commercially available mammalian-derived transglutaminase serving as a reagent, such as guinea pig liver-derived transglutaminase, goat-derived transglutaminase, or rabbit-derived transglutaminase produced by, for example, Oriental Yeast Co., Ltd., Upstate USA Inc., and Biodesign International; and human-derived blood coagulation factors (e.g., Factor XIIIa, Haematologic Technologies, Inc.).

Crosslinking of a recombinant gelatin includes a step of mixing a biopolymer solution and a crosslinking agent and a step of allowing the obtained homogeneous solution to react.

According to the present invention, the mixing temperature for treating a biopolymer with a crosslinking agent is not particularly limited as long as the resulting solution can be uniformly stirred. The temperature is preferably 0° C. to 40° C., more preferably 0° C. to 30° C., further preferably 3° C. to 25° C., still further preferably 3° C. to 15° C., even further preferably 3° C. to 10° C., and particularly preferably 3° C. to 7° C.

After mixing of a biopolymer and a crosslinking agent, the temperature can be increased. The reaction temperature is not particularly limited as long as the crosslinking reaction progresses. However, in view of denaturation or degradation of the biopolymer, the temperature is substantially 0° C. to 60° C., preferably 0° C. to 40° C., more preferably 3° C. to 25° C., further preferably 3° C. to 15° C., still further preferably 3° C. to 10° C., and particularly preferably 3° C. to 7° C.

The form of the construct used as a coating material in the present invention is not particularly limited. Such construct can be in the form of sponge, film, nonwoven fabric, fiber (tube), particles, mesh, or the like. Such construct can be applied in any shape. However, the construct is, for example, a pyramid construct, a conical construct, a prismatic construct, a cylindrical construct, a globular construct, a spindle construct, or a construct formed with an arbitrary mold. Preferably, the construct is a prismatic construct, a cylindrical construct, a spindle construct, or a construct formed with an arbitrary mold. More preferably, the construct is a pyramid construct, a conical construct, a prismatic construct, or a cylindrical construct. Most preferably, the construct is a prismatic construct or a cylindrical construct.

The size of the construct is not particularly limited. However, when it is in the form of sponge or nonwoven fabric, the size is preferably a 500-cm square or less, more preferably a 100-cm square or less, particularly preferably a 50-cm square or less, and most preferably a 10-cm square or less. In the case of a fiber (tube), the diameter (or one side of the circumference) of a fiber or a tube is 1 nm to 10 cm, preferably 1 nm to 1 cm, more preferably 1 nm to 100 μm, particularly preferably 1 nm to 1 μm, and most preferably 1 nm to 10 nm. In addition, the length thereof is not particularly limited. However, the length is preferably 10 μm to 100 m, more preferably 100 μm to 10 m, further preferably 1 mm to 1 m, and most preferably 1 cm to 30 cm. In the case of particles, the particle size is preferably 1 nm to 1 mm, more preferably 10 nm to 200 μm, further preferably 50 nm to 100 μm, and particularly preferably 100 nm to 10 μm.

The thickness of a construct is not particularly limited. However, the thickness is preferably 1 nm or more, more preferably 10 nm or more, further preferably 100 nm or more, still further preferably 1 μm or more, even further preferably 10 μm or more, and most preferably 100 μm or more.

A solvent used for preparing the above construct is not particularly limited. However, such solvent is preferably water or an organic fluorine compound; more preferably water or an organic fluorine compound having 10 or less carbon atoms; more preferably water or an organic fluorine compound having 10 or less carbon atoms and containing an ester group, an ether group, a ketone group, a carboxylic acid group, a cyano group, a hydroxyl group, a phenol group, a benzyl group, a vinyl group, chloride, and bromide; further preferably water, 1,1,1,3,3,3-hexafluoro-2-propanol (HFIP), 2,2,2-trifluoroethanol (TFE), hexafluoroacetone, trifluoroacetic acid, or pentafluoropropionic acid; still further preferably water, HFIP, TFE, or hexafluoroacetone; and most preferably water, HFIP or TFE.

The above solvents can be used as mixed solvents. The content of water or an organic fluorine compound in a mixed solvent that can be used is preferably 1% or more, more preferably 10% or more, further preferably 50% or more, and most preferably 80% or more.

The recombinant gelatin may be used in a mixture containing another synthetic polymer. Preferred examples of such synthetic polymer include polylactic acid, polyglycolic acid, a copolymer of either thereof, poly(ε-caprolacton), poly(hydroxyalkanoate) (PHA), polyethyleneterephthalate (PET), polyurethane, polymethylenecarbonate, glycerol, polyethyleneglycol, hyaluronic acid benzyl ester, hyaluronic acid ethyl ester, and acetylcellulose.

The material of a substrate to be coated is not particularly limited. However, the material is substantially a metal, a synthetic polymer, or a biopolymer. Preferably, the material is nickel, iron, titanium, polyester, polyamide, polyurethane, polycarbonate, polyether, vinylpolymer, a fluorine-containing polymer, polypeptide, or polysaccharide; more preferably nickel, iron, titanium, polylactic acid, polyglycolic acid, a copolymer of any thereof, poly(ε-caprolacton), poly(hydroxyalkanoate) (PHA), polyethyleneterephthalate (PET), polyurethane, polymethylenecarbonate, glycerol, polyethyleneglycol, hyaluronic acid benzyl ester, hyaluronic acid ethyl ester, acetylcellulose, polystyrene, polyvinyl alcohol, polytetrafluoroethylene, collagen, gelatin, or albumin. More preferably, the material is PET, polyurethane, segmented polyurethane, polytetrafluoroethylene, or a collagen. A single such material may be used alone, or combinations of the above materials may be used.

The molecular weight of the synthetic polymer is not particularly limited. However, the molecular weight is substantially 1 KDa to 10 MDa, preferably 5 KDa to 500 KDa, and most preferably 10 KDa to 100 KDa. Further, the synthetic polymer may be crosslinked or chemically modified.

An additive may be added to the recombinant gelatin used in the present invention according to need. Examples of additives include drugs, coloring agents, softening agents, moisturizing agents, thickeners, surfactants, preservatives, fragrant materials, and pH adjusters.

The coating material used for a biomedical material surface of the present invention may include a drug. Such drug is a physiologically active ingredient. Specific examples thereof include percutaneous absorbents, topical therapeutic agents, oral therapeutic agents, cosmetic ingredients, and supplement ingredients. Specific examples of a drug include anti-inflammatory agents, antibacterial agents, antibiotics, immunosuppressive agents, antioxidants, anticancer agents, vitamins, nucleic acids, and antibodies. Particularly preferred examples thereof are anti-inflammatory agents. Both steroidal and nonsteroidal anti-inflammatory agents may be used. Examples of anti-inflammatory agents include aspirin, acetaminophen, phenacetin, indomethacin, diclofenac sodium, piroxicam, fenoprofen calcium, ibuprofen, chlorpheniramine maleate, diflunisal, dexamethasone sodium phosphate, paclitaxel, docetaxel, 5-fluorouracil, Topotecin, cisplatin, rapamycin, tacrolimus, and cyclosporin. Vitamins that can be used may be water-soluble vitamins or fat-soluble vitamins. Examples of such vitamins include vitamin A, the vitamin B group, vitamin C, the vitamin D group, vitamin E, and vitamin K. Specific examples of drugs are described above. However, as long as the recombinant gelatin used in the present invention is applied, drugs that can be used are not limited to the above drugs.

Drug inclusion can be carried out simultaneously with coating of a biomedical material surface or after such coating. For instance, coating may be carried out with the use of a recombinant gelatin solution containing a drug such that the drug can be included in the obtained coating layer. In addition, according to an alternative method, a recombinant gelatin coating layer may be prepared and then immersed in a solution containing a drug such that the drug can be included in the coating layer.

The present invention is hereafter described in greater detail with reference to the following examples, although the technical scope of the present invention is not limited thereto.

### EXAMPLES

### Example 1

### Synthesis of Recombinant Gelatins

Recombinant gelatins having different properties were synthesized in accordance with EP-A-0926453, EP-A-1014176, and WO01/34646. A gelatin (RGD-gelatin (represented by SEQ ID NO: 2 of WO2004/085473)) having an RGD sequence (a minimal amino acid sequence functioning as a cell adhesion signal) and gelatins having no RGD sequence (HU, HU3, HU4, P, and P4, collectively referred to as “n-RGD-gelatin”) were obtained.

Name: HU

(SEQ ID NO: 1)

GPPGEPGPTGLPGPPGERGGPGSRGFPGADGVAGPKGPAGERGSPGPAGP
KGSPGEAGRPGEAGLPGAKGLTGSPGSPGPDGKTGPPGPACQDGRPGPPG
PPGARGQAGVMGFPGPKGAAGEPGKAGERGVPGPPGAVGPAGKDGEAGAQ
GPPGPAGPAGERGEQGPAGSPGFQGLPGPAGPPGEAGKPGEQGVPGDLGA
PGPSGPA
Name: HU3

(SEQ ID NO: 2)

GPPGEPGPTGLPGPPGERGGPGSRGFPGADGVAGPKGPAGERGSPGPAGP
KGSPGEAGRPGEAGLPGAKGLTGSPGSPGPDGKTGPPGPACQDGRPGPPG
PPGARGQAGVMGFPGPKGAAGEPGKAGERGVPGPPGAVGPAGKDGEAGAQ
GPPGPAGPAGERGEQGPAGSPGFQGLPGPAGPPGEAGKPGEQGVPGDLGA
PGPSGPAGEPGPTGLPGPPGERCGPGSRGFPGADGVAGPKGPAGERGSPG
PAGPKGSPGEAGRPGEAGLPGAKGLTGSPGSPGPDGKTGPPGPAGQDGRP
GPPGPPGARCQAGVMGFPGPKGAAGEPGKAGERGVPGPPGAVGPAGKDGE
AGAQGPPGPAGPAGERGEQGPAGSPGFQGLPGPAGPPGEAGKPGEQGVPG
DLGAPGPSGPAGEPGPTGLPGPPGERCGPGSRGFPGADGVAGPKGPAGER
GSPGPAGPKGSPGEAGRPGEAGLPGAKGLTGSPGSPGPDGKTGPPGPAGQ
DGRPGPPGPPGARCQAGVMGFPGPKGAAGEPGKAGERGVPGPPGAVGPAG
KDGEAGAQGPPGPAGPAGERGEQGPAGSPGFQGLPGPAGPPGEAGKPGEQ
GVPGDLGAPGPSGPAG
Name: HU4

(SEQ ID NO: 3)

GPPGEPGPTGLPGPPGERGGPGSRGFPGADGVAGPKGPAGERGSPGPAGP
KGSPGEAGRPGEAGLPGAKGLTGSPGSPGPDGKTGPPGPACQDGRPGPPG
PPGARGQAGVMGFPGPKGAAGEPGKAGERGVPGPPGAVGPAGKDGEAGAQ
GPPGPAGPAGERGEQGPAGSPGFQGLPGPAGPPGEAGKPGEQGVPGDLGA
PGPSGPAGEPGPTGLPGPPGERCGPGSRGFPGADGVAGPKGPAGERGSPG
PAGPKGSPGEAGRPGEAGLPGAKGLTGSPGSPGPDGKTGPPGPAGQDGRP
GPPGPPGARCQAGVMGFPGPKGAAGEPGKAGERGVPGPPGAVGPAGKDGE
AGAQGPPGPAGPAGERGEQGPAGSPGFQGLPGPAGPPGEAGKPGEQGVPG
DLGAPGPSGPAGEPGPTGLPGPPGERCGPGSRGFPGADGVAGPKGPAGER
GSPGPAGPKGSPGEAGRPGEAGLPGAKGLTGSPGSPGPDGKTGPPGPAGQ
DGRPGPPGPPGARCQAGVMGFPGPKGAAGEPGKAGERGVPGPPGAVGPAG
KDGEAGAQGPPGPAGPAGERGEQGPAGSPGFQGLPGPAGPPGEAGKPGEQ
GVPGDLGAPGPSGPAGEPGPTGLPGPPGERGGPGSRGFPGADGVAGPKGP
AGERGSPGPAGPKGSPGEAGRPGEAGLPGAKGLTGSPGSPGPDGKTGPPG
PACQDGRPGPPGPPGARGQAGVMGFPGPKGAAGEPGKAGERGVPGPPGAV
GPAGKDGEAGAQGPPGPAGPAGERGEQGPAGSPGFQGLPGPAGPPGEAGK
PGEQGVPGDLGAPGPSGPACG
Name: P

(SEQ ID NO: 4)

GPPGEPGNPGSPGNQGQPGNKGSPGNPGQPGNEGQPGQPGQNGQPGEPGS
NGPQGSQGNPGKNGQPGSPGSQGSPGNQGSPGQPGNPGQPGEQGKPGNQG
PAGG
Name: P4

(SEQ ID NO: 5)

GPPGEPGNPGSPGNQGQPGNKGSPGNPGQPGNEGQPGQPGQNGQPGEPGS
NGPQGSQGNPGKNGQPGSPGSQGSPGNQGSPGQPGNPGQPGEQGKPGNQG
PAGEPGNPGSPGNQGQPGNKGSPGNPGQPGNEGQPGQPGQNGQPGEPGSN
GPQGSQGNPGKNGQPGSPGSQGSPGNQGSPGQPGNPGQPGEQGKPGNQGP
AGEPGNPGSPGNQGQPGNKGSPGNPGQPGNEGQPGQPGQNGQPGEPGSNG
PQGSQGNPGKNCQPGSPGSQGSPGNQGSPCQPGNPGQPGEQGKPGNQGPA
GEPGNPGSPGNQGQPGNKGSEGQPGQPGQNGQPGEPGSNGPQGSQGNPGK
NGQPGSPGSQGSPGNQGSPCQPGNPGQPGEQGKPGNQGPAGG

### Example 2

### Preparation of Gelatin-Crosslinked Films

An aqueous solution (5 mL) containing an acid-treated pig skin gelatin (PSK gelatin, Nippi, Inc.) or a recombinant gelatin (HU4, CBT, P, P4, or RGD-gelatin) (0.26%) and a compound (CH₂═CH—SO₂—(CH₂)₂—SO₂—CH═CH₂) (0.016%) was introduced into a container (12 cm×7 cm) and allowed to stand overnight. Thus, a crosslinked gelatin film (film 1) was prepared.

Meanwhile, a PBS solution (5 mL) containing glutaraldehyde (0.002%) and the above gelatin (0.1%) was introduced into a container (12 cm×7 cm) and allowed to stand overnight. Thus, a crosslinked film was obtained. The obtained crosslinked film was immersed in a 50 mM glycine solution such that unreacted aldehyde groups were deactivated, followed by washing with water. Accordingly, a glutaraldehyde-crosslinked gelatin film (film 2) was obtained.

### Example 3

### Cell Adhesiveness of Crosslinked Gelatin Films

Each film prepared in Example 2 (film 1) was cut into square pieces (1.2×1.2 cm) and placed in a 12-well cell culture dish. Bovine vascular endothelial cells were seeded on the film pieces (cell density: 1.0×10⁴ cells/mL, 1 mL) and cultured in an incubator. Cells obtained 3 hours thereafter and on Days 1, 3, and 7 were observed with a phase-contrast microscope. The cells did not adhere to the HU4, P, and P4 films. On the other hand, cell adhesion was observed on the surface of the RGD-gelatin film and that of the acid-treated gelatin film. Therefore, it can be said that HU4, P, and P4 can be used for a non-cell-adhesive matrix and that the RGD-gelatin can be used for a cell-adhesive matrix.

Meanwhile, the results obtained with the use of the glutaraldehyde-crosslinked gelatin film (film 2) were similar to those obtained with the use of the above film 1.

TABLE 1
Cell adhesiveness of gelatin

| Gelatin | Cell adhesiveness |
|---|---|
| HU4 | Not observed |
| RGD-gelatin | Observed |
| P | Not observed |
| P4 | Not observed |
| Acid-treated pig skin gelatin | Observed |

### Example 4

### Antithrombogenicity of Films Covered with Vascular Endothelial Cells

Human blood was added dropwise onto each of the gelatin films covered with vascular endothelial cells prepared in Example 3 (RGD-gelatin film and acid-treated pig skin film). The time period until the completion of coagulation was measured for evaluation of antithrombogenicity. In the case of the film prepared from the RGD gelatin, significant antithrombogenicity was observed as compared with the acid-treated pig skin gelatin.

### Example 5

### In Vivo Degradation

A PBS solution containing a recombinant gelatin (HU3, HU4, RGD-gelatin, P4, V3, or fibrogen-100 Kda (RhG100-001, FibroGen)) or a pig skin gelatin (PSP gelatin, Nippi, Inc.) (10%) was mixed with glutaraldehyde (0.4%). The resultant (1 mL) was added to a 1.5-mL tube (φ=8 mm) and allowed to stand overnight. Thus, a gelatin gel was prepared. 400 μL of thermolysin (concentration: 5 μM) was added to the tube. The tube was rotated with a rotator. Then, the decrease in the gel height after the elapse of a certain period of time was measured. Except for the case of P4, gel was degraded in each case with the elapse of time. Except for the case of the RGD-gelatin, degradation was observed at similar levels in the cases of each recombinant gelatin and the pig skin gelatin. Degradation was observed to a greater extent in the case of the RGD-gelatin than in the case of the pig skin gelatin.

It can be said that it is possible to control degradation depending on the type of recombinant gelatin. That is, it is possible to control degradation in accordance with a desired application.

### Example 6

### Drug Inclusion

A 1,1,1,3,3,3-hexafluoro-2 propanol (HFIP) solution or a 2,2,2-trifluoroethanol (TFE) solution containing a different one of the above recombinant gelatins (100 mg/mL) and paclitaxel (1 mg/mL) was applied to polypropylene (20 cm×10 cm, thickness: 1 mm). The obtained film was allowed to stand in a dryer (temperature: 50° C., humidity: 95%) for 7 days, followed by natural drying for 3 days for removal of a solvent. Thus, a gelatin film containing paclitaxel was obtained.

The HFIP and TFE amounts in such film accounted for 0.001% or less of the gelatin weight. In addition, paclitaxel used in a drug-releasing stent is a hydrophobic drug. Thus, it is generally difficult for paclitaxel to be included in a hydrophilic substrate. However, paclitaxel included in the film did not deposit and remained finely dispersed therein.

### Example 7

### Preparation of Paclitaxel-Containing Films

An HFIP solution containing paclitaxel (1 mg/mL) was coated to each crosslinked recombinant gelatin film prepared in Example 2 (coating thickness: 1 mm) such that the film was immersed in the solution. Accordingly, paclitaxel was included in each film. HFIP was removed in the manner described in Example 6. Thus, crosslinked gelatin films containing paclitaxel were obtained.

### Example 8

### Cell Adhesiveness on Drug-Containing Films

Bovine aorta-derived vascular smooth muscle cells were seeded on each crosslinked gelatin film containing paclitaxel prepared in Example 7, followed by culturing. The cells did not adhere to any of the films and died.

### Example 9

### Coating of Synthetic Polymer Surface

HFIP solution (0.1%) samples each containing a different one of the above recombinant gelatins was coated to a segmented polyurethane or polyethyleneterephthalate (PET) film known as a material for artificial blood vessels (coating thickness: 1 mm), followed by natural drying for 1 hour. Then, HFIP was removed in the manner described in Example 6 and thus a recombinant-gelatin-coated film was obtained. The water contact angle of each film was measured. As a result, a recombinant-gelatin-coated film was found to have hydrophilized to a greater extent in each case involving the use of a different gelatin than the cases involving the use of an untreated film or an HFIP-treated film.

### Example 10

### Coating of Metal Surface

A recombinant-gelatin-coated surface was prepared by coating HFIP solution (0.1%) or aqueous solution (0.1%) samples each containing a different one of the above recombinant gelatins to the surface of a stainless-steel stent (coating thickness: 1 mm) and drying the surface.

It can be said that it is possible to control cell adhesiveness/degradation on a biomedical material with the use of a recombinant gelatin. Thus, control of drug inclusion and coating with high biocompatibility can be realized.

1. A coating material for a surface of a biomedical material, which comprises a recombinant gelatin.

2. The coating material according to claim 1, wherein the recombinant gelatin has a homology of 80% or more with the amino acid sequence of a natural collagen.

3. The coating material according to claim 1, wherein the recombinant gelatin comprises GXY triplets that are characteristic of collagen and has a molecular weight of 2 KDa to 100 KDa.

4. The coating material according to claim 1, wherein the recombinant gelatin comprises GXY triplets that are characteristic of collagen and has a molecular weight of 10 KDa to 90 KDa.

5. The coating material according to claim 1, wherein the recombinant gelatin comprises repeats of a partial sequence of a natural collagen.

6. The coating material according to claim 1, wherein the biomedical material is a material used in vivo.

7. The coating material according to claim 1, wherein the biomedical material is a material used for intravascular treatment.

8. The coating material according to claim 1, wherein the biomedical material is a stent or an artificial blood vessel.

9. The coating material according to claim 1, wherein the surface of a biomedical material is formed with segmented polyurethane, polyethyleneterephthalate, polylactic acid, polyglycolic acid, a lactic acid-glycol acid copolymer, stainless steel, or nickel.

10. The coating material according to claim 1, in which a drug is contained.

11. The coating material according to claim 10, wherein the drug is an anti-inflammatory agent, an antibacterial agent, an antibiotic, an anticancer agent, or an immunosuppressive agent.

12. The coating material according to claim 10, wherein the drug is an anticancer agent or an immunosuppressive agent.

13. The coating material according to claim 10, wherein the drug is paclitaxel, docetaxel, cisplatin, rapamycin, or tacrolimus.

14. The coating material according to claim 10, wherein the drug is a cytokine, a hormone, a polypeptide, or a nucleic acid.

15. The coating material according to claim 1, wherein the recombinant gelatin is crosslinked.

16. The coating material according to claim 15, wherein the crosslinking is carried out with the use of an aldehyde, a condensing agent, or an enzyme.

17. The coating material according to claim 1, wherein an organic fluorine compound is used for inclusion of the drug or coating of the surface of a biomedical material.


SEQUENCE LISTING (13 sequences)

SEQ ID NO: 1 - protein, 207 aa, Artificial Sequence
  Synthetic Recombinant Gelatin
GPPGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPA

SEQ ID NO: 2 - protein, 616 aa, Artificial Sequence
  Synthetic Recombinant Gelatin
GPPGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAG

SEQ ID NO: 3 - protein, 821 aa, Artificial Sequence
  Synthetic Recombinant Gelatin
GPPGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGPAGG

SEQ ID NO: 4 - protein, 104 aa, Artificial Sequence
  Synthetic Recombinant Gelatin
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 5 - protein, 401 aa, Artificial Sequence
  Synthetic Recombinant Gelatin
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 6 - protein, 4 aa, Artificial Sequence
  Synthetic Cell adhesion peptide
REDV

SEQ ID NO: 7 - protein, 5 aa, Artificial Sequence
  Synthetic Cell adhesion peptide
YIGSR

SEQ ID NO: 8 - protein, 5 aa, Artificial Sequence
  Synthetic Cell adhesion peptide
PDSGR

SEQ ID NO: 9 - protein, 7 aa, Artificial Sequence
  Synthetic Cell adhesion peptide
RYVVLPR

SEQ ID NO: 10 - protein, 6 aa, Artificial Sequence
  Synthetic Cell adhesion peptide
LGTIPG

SEQ ID NO: 11 - protein, 10 aa, Artificial Sequence
  Synthetic Cell adhesion peptide
RNIAEIIKDI

SEQ ID NO: 12 - protein, 5 aa, Artificial Sequence
  Synthetic Cell adhesion peptide
IKVAV

SEQ ID NO: 13 - protein, 4 aa, Artificial Sequence
  Synthetic Cell adhesion peptide
DGEA